(19) United States
(12) Patent Application Publication
(10) Pub. No.: US 2021/0128863 A1
(43) Pub. Date: May 6, 2021

(54) NASAL CANNULA AND TUBING WITH VENTILATOR SYSTEM

(71) Applicant: Neotech Products LLC, Valencia, CA (US)

(72) Inventors: Matthew McNally, Sunapee, NH (US); Richard Mitchell, Fairlee, VT (US); Lawrence Kiliszewski, The Woodlands, TX (US); Craig McCrary, Valencia, CA (US)

(73) Assignee: Neotech Products LLC, Tokyo (JP)

(21) Appl. No.: 17/150,755

(22) Filed: Jan. 15, 2021

Related U.S. Application Data
(63) Continuation-in-part of application No. 16/436,615, filed on Jun. 10, 2019.
(60) Provisional application No. 62/705,019, filed on Jun. 7, 2020.

(51) Int. Cl. A61M 16/06; A61M 16/08; A61M 16/00; A61M 16/10; A61M 16/16

(52) U.S. Cl. CPC A61M 16/0666; A61M 16/0816; A61M 16/16; A61M 16/1075; A61M 16/0057

(57) ABSTRACT

A nasal cannula system for use in ventilator systems, particularly continuous positive airway pressure (CPAP) systems. The nasal cannula system has a nasal body with nare prongs that extend into a patient's nostrils when worn. The ventilator system includes a ventilator on an inhalation side and either a bubble chamber or a variable resistance valve on the exhalation side. The inhalation side may include a heater/humidifier in-line with the supply tube so as to treat the air before application. The airway tube may have a generally semi-circular cross-section so as to provide a flat exterior surface for abutment or contact with a patient's skin.

### RELATED APPLICATIONS

This application is a continuation-in-part of U.S. application Ser. No. 16/436,615 filed on Jun. 10, 2019. This application also claims the benefit of U.S. Provisional Application No. 62/705,019 filed on Jun. 7, 2020.

### BACKGROUND OF THE INVENTION

The present invention is directed to airway delivery systems used in medical treatment, and more particularly to tubing and connections for the use with a ventilator system having a configuration to facilitate such systems. The shape of the tubing and connections is intended to provide a more comfortable and stable placement of the airway delivery systems on and around a patient, particularly their face. The configuration of the ventilator system is designed to facilitate use of a ventilator on a patient while utilizing the novel nasal cannula and tubing.

Nasal continuous positive airway pressure, NCPAP, is a standard used for administration of non-invasive positive airway pressure, particularly in the Neonate. Administration of non-invasive positive airway pressure is usually accomplished by tubing being run from behind or above a patient's head to their nose where cannula are inserted into the nasal openings. The tubing is generally run above a patient's ears and across the cheeks.

Typical tubing and connections for such systems have a circular cross-section. Alternative systems may utilize tubing having an oval cross-section, or a semi-circular or D-shaped cross-section, with or without an internal rib.

Such nasal cannula and tubing ventilator systems would be beneficial in treatments requiring less invasive positive pressure treatment means. The nasal cannula system is less intrusive and less obstructive than traditional CPAP masks. Such novel systems can oftentimes be more comfortable and more convenient for treatment, especially for infant patents or others that are less able to receive and follow treatment instructions.

Accordingly, there is a need for tubing and connections for use in airway delivery systems that minimize the risk of sliding, moving, dislodging, and pinching through patient movement during use, particularly in neonatal patients. The present invention fulfills these needs and provides other related advantages.

### SUMMARY OF THE INVENTION

The present invention is directed to a nasal cannula system having a nasal body with a pair of nasal prongs configured for insertion into nasal passages when worn by a patient. A nasal cannula system, having a nasal prong with nare tubes ending in nare ports configured for insertion into the nostrils of a patient. A first airway limb is attached to a first connector on the nasal prong and in fluid communication with the nare tubes. A second airway limb is attached to a second connector on the nasal prong and in fluid communication with the nare tubes. The first airway limb comprises an inhalation tube on a ventilator system. The second airway limb comprises an exhalation tube on the ventilator system.

The inhalation tube is operationally connected to a positive pressure port on a ventilator. The exhalation tube is operationally connected to a back pressure device configured to maintain continuous positive pressure in the nasal cannula system, specifically in the patient's lungs. The back pressure device may be either a bubble chamber or a variable control valve port on the ventilator. The system may further include a heater-humidifier fluidly disposed in the inhalation tube between the ventilator and the nasal prong. In addition, a wedge elevating pad may be included for disposal underneath a patient wearing the nasal prong.

The first airway limb and the second airway limb may both have a generally flat side joined to a generally curved side that together form a passageway therebetween having a semi-circular cross-section; and an internal rib extending along a length of the passageway, spanning from the generally flat side to the generally curved side. The internal rib may be continuous along the length of the passageway so as to divide each of the first airway limb and the second airway limb into two separate passageways. Alternatively, the internal rib may be discontinuous along the length of the passageway so as to allow fluid communication between regions of the passageway on opposite sides of the internal rib. The first airway limb and the second airway limb may both have a generally flat exterior side configured to lay against a patient's skin when worn.

Other features and advantages of the present invention will become apparent from the following more detailed description, taken in conjunction with the accompanying drawings, which illustrate, by way of example, the principles of the invention.

### BRIEF DESCRIPTION OF THE DRAWINGS

The accompanying drawings illustrate the invention. In such drawings:

FIG. 1 is an environmental view illustrating an application of a first preferred embodiment of the inventive nasal cannula system as attached to an infant patient;

FIG. 2 is a perspective view of the first preferred embodiment of the inventive nasal cannula system;

FIG. 3 is a close-up perspective view of the connection between a tube and a connector of a preferred embodiment of the inventive nasal cannula system;

FIG. 4 is a perspective view of the inventive nasal cannula system;

FIG. 4A is a partial cut-away of a first preferred embodiment of the airway tube of the inventive nasal cannula system;

FIG. 4B is a partial cut-away of a second preferred embodiment of the airway tube of the inventive nasal cannula system;

FIG. 4C is a partial cut-away of a third preferred embodiment of the airway tube of the inventive nasal cannula system;

FIG. 5 is a perspective transparent view of a length of a first preferred embodiment of the internal rib in the gas supply tube;

FIG. 6 is a perspective transparent view of a length of a second preferred embodiment of the internal rib in the gas supply tube;

FIG. 7A is a perspective view of a length of gas supply tube being inserted into a supply adapter;

FIG. 7B is a cut-away perspective view of a length of gas supply tube being inserted into a supply adapter;

FIG. 8 is an environmental view of the inventive nasal cannula system utilized in a ventilator system;

FIG. 8A is a close-up of the inventive nasal cannula system in FIG. 8;

FIG. 9 is an illustration of the application of the inventive nasal cannula system to a patient;

FIG. 10 is an illustration of the connection of the inventive nasal cannula system to inhalation and exhalation tubing;

FIG. 11 is an environmental view of the inventive nasal cannula system utilized in a ventilator system; and

FIG. 12 is an environmental view of the inventive nasal cannula system utilized in another ventilator system.

### DETAILED DESCRIPTION OF THE PREFERRED EMBODIMENTS

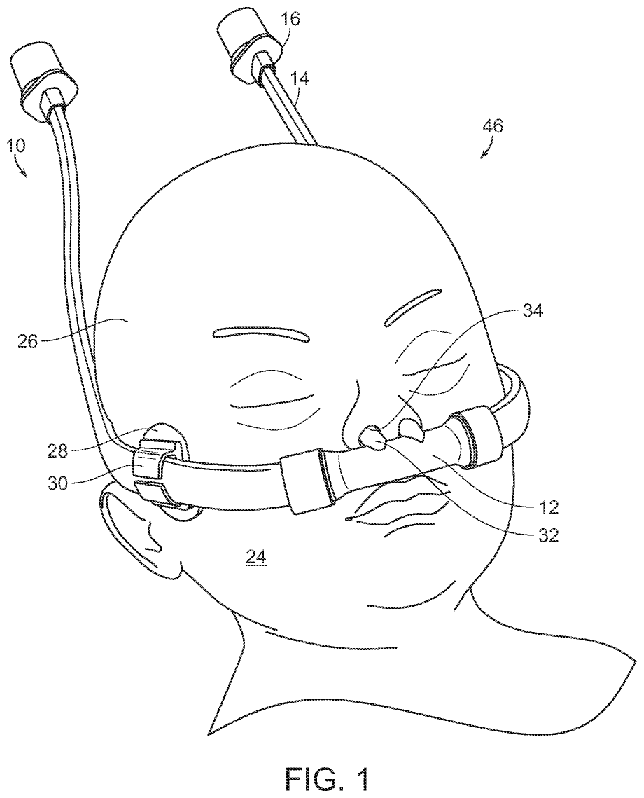

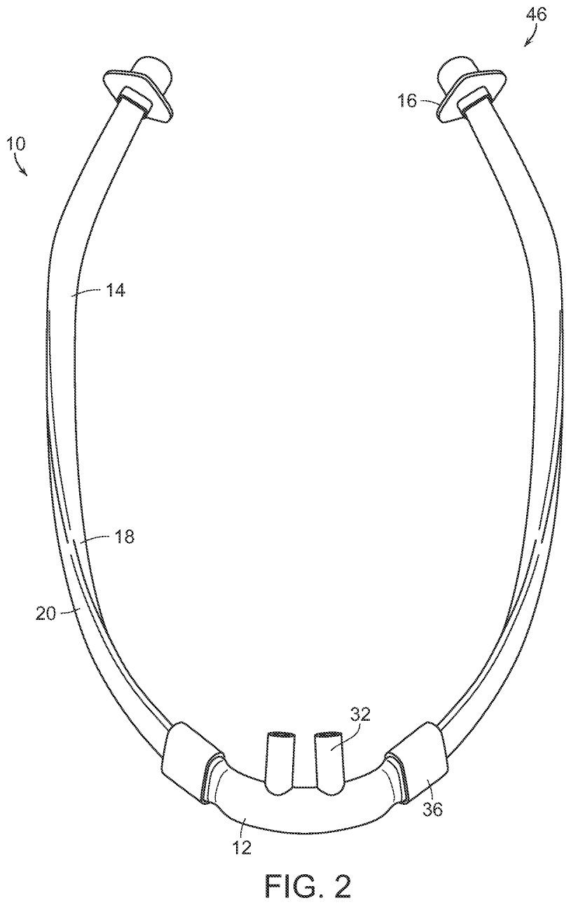

In the following detailed description, the nasal cannula system of the present invention is generally referred to by reference numeral 10 in FIGS. 1-4. The primary components of the system 10 represent the nasal body 12, the airway tubing 14, and the supply adapter 16.

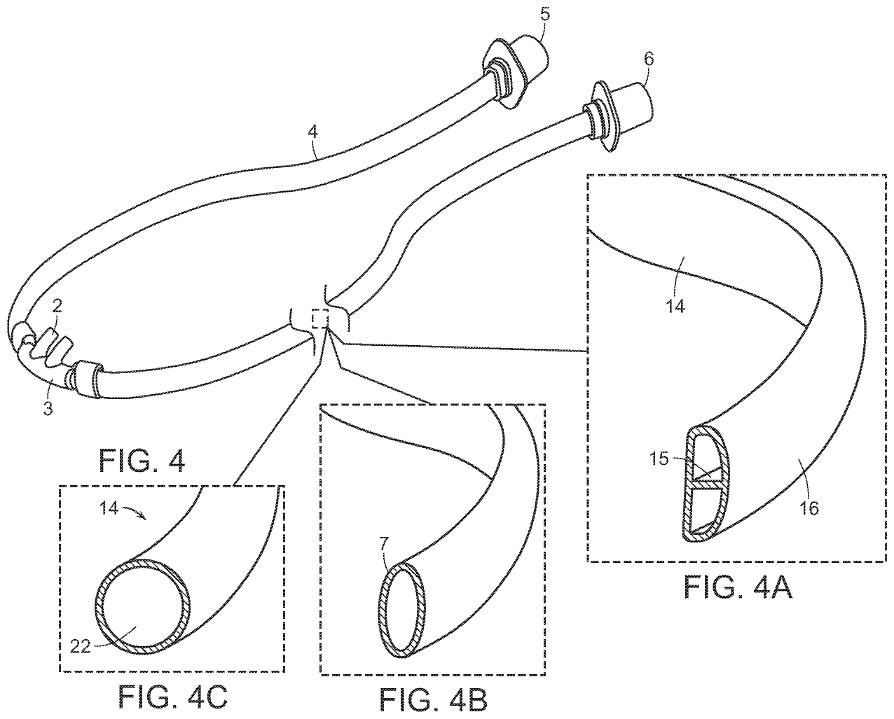

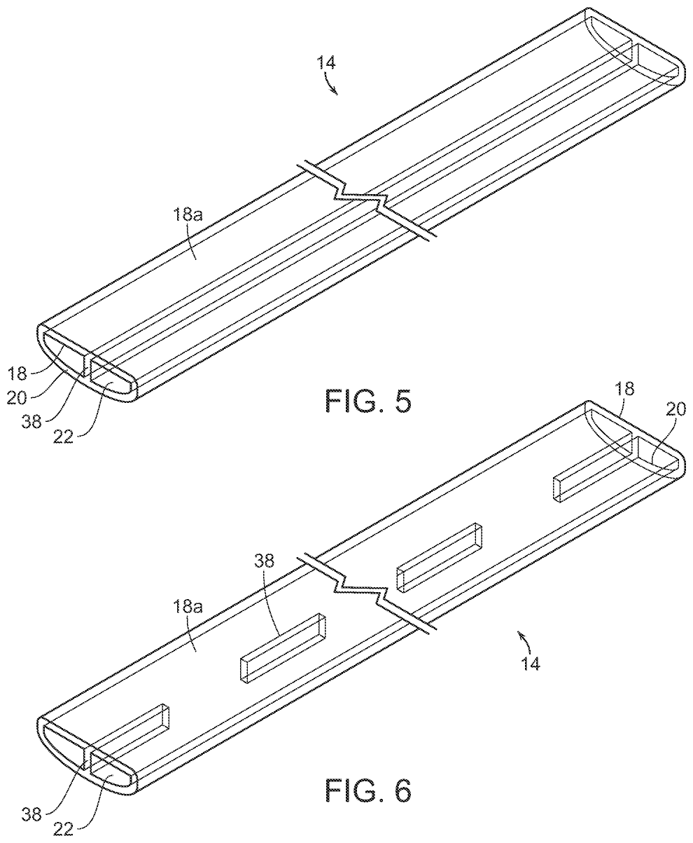

The airway tubing 14, shown in cross-section in FIG. 4A and in transparent form in FIGS. 5 and 6, preferably has a generally semi-circular or D-shaped cross-section. The airway tubing 14 may also come in other cross-sectional shapes. FIG. 4B shows an alternate embodiment for the airway tubing 14 that has an oval cross-section. FIG. 4C shows another alternate embodiment for the airway tubing 14 that has an oval cross-section. FIG. 4D shows another alternate embodiment for the airway tubing 14 that has a circular cross-section. Such oval cross-section presents similar benefits to those of the semi-circular cross-section described below.

The semi-circular cross-sectional tubing 14 has a generally flat side 18 and a generally curved side 20 that a connected to form a passageway 22 having the semi-circular cross-section. The flat side 18 is flat on at least an exterior surface 18a relative to the tubing 14, but is preferably flat on an interior surface as well. Similarly, the curved side 20 is curved on at least an exterior surface 20a relative to the tubing 14, but is preferably curved on an interior surface as well.

When the flat side 18 and curved side 20 are combined, the exterior surfaces 18a, 20a combined to create a generally semi-circular cross-section on the exterior of the tubing 14. Similarly, when the interior surfaces of the flat side 18 and curved side 20 are flat and curved, respectively, they form a passageway 22 through the tubing 14 that has a semi-circular cross-section.

The flat exterior surface 18a of the flat side 18 is configured to lay flush against the skin of a patient when the tubing 14 is used. In this way, the tubing 14 has a lower profile when resting against the skin 24 of a patient 26, e.g., on the cheek or otherwise around the face or head, as shown in FIG. 1. This lower profile minimizes the degree to which a patient, particularly an unconscious or an infant patient, may disturb or dislodge the tubing 14 or system 10 due to interference from hand or arm movement near the tubing 14 or contact with other objects, e.g., pillows, due to movement of the head. The lower profile and exposed exterior curved surface 20a also minimizes pinching or pressure points when the tubing 14 may be pressed between the patient's skin and an external object, i.e., a pillow or other medical device.

The flat exterior surface 18a also minimizes the degree to which the inventive tubing 14 may roll, slide, or otherwise be displaced when in use on a patient. The flat exterior surface 18a provides a stable surface with increased contact against the skin 24 of a patient. Such stable surface minimizes rolling and the increased contact minimizes sliding or other movement across the skin 24. To assist in this effort, the system 10 may include a surface retainer 28 that is configured to removably adhere to the skin 24 of a patient 26. The surface retainer has a clamp portion 30 is slightly raised above the skin 24 and has a semi-circular cross-section that generally matches the semi-circular cross-section of the tubing 14.

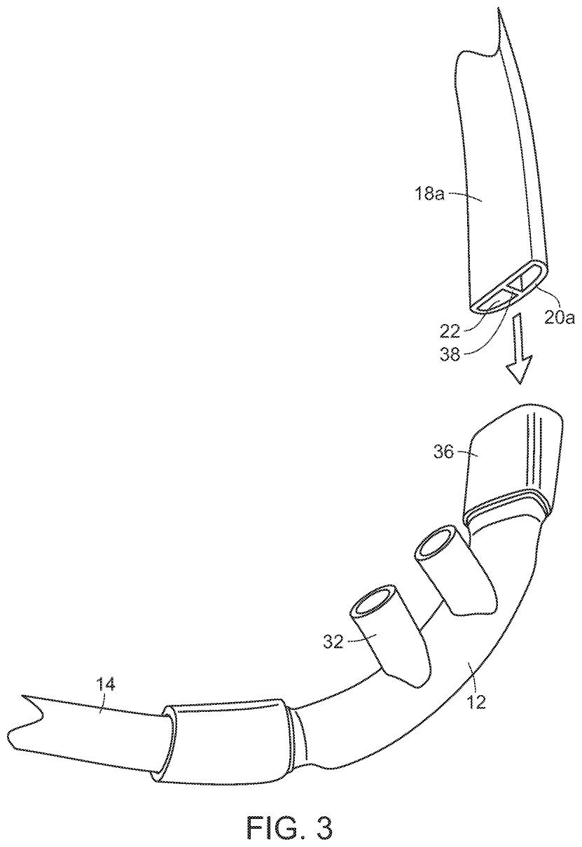

As shown in FIG. 3, the nasal body 12 comprises an elongated tube and preferably has a flat exterior surface 12a that is positioned so as to lay flush against the patient's skin 14, e.g., on the upper lip beneath the nose. The nasal body 12 includes a pair of nasal prongs 32 that are configured to enter nasal openings 34 when in use. The nasal body 12 also includes at least one tube connector 36 at one end of the body 12 for receipt of airway tubing 14. The tube connector 36 also has a generally flat exterior surface 36a that matches the flat exterior surface 12a of the nasal body 12. The tube connector 36 also preferably has a semi-circular cross-section that matches the semi-circular cross-section of the airway tubing 14. Where the airway tubing 14 has a shape other than semi-circular, the tube connectors 36 have a cross-section to match the airway tubing 14.

Where the nasal body 12 has a single tube connector 36 at one end, the other end of the nasal body is closed off. In this way, airway tubing 14 can introduce oxygen or another gas into the nasal body 12 for passage through the nasal prongs 32 into the nasal openings 34. Preferably, the nasal body 12 has tube connectors 36 opposite ends thereof, each having a generally semi-circular cross-section configured for sliding reception of inventive airway tubing 14. In this way, oxygen or other gases can be introduced into the nasal body 12 for administration to the patient 26. The airway tubing 14 may include an inhalation tube 14a wherein fluid flows toward the nasal body 12 and an exhalation tube 14b wherein fluid flows away from the nasal body 12.

The tubing 14 preferably has an internal rib 38 that runs the length of the tubing 14 through the passageway 22. The internal rib 38 is designed to provide additional rigidity to the tubing 14 such that the passageway 22 does not become completely closed off or otherwise blocked when the patient 26 may lay on the tubing 14 or other object exerts pressure on the exterior of the tubing 14. The internal rib 38 may be continuous so as to completely divide the passageway 22 into two separate passageways.

Alternatively, and preferably, the internal rib 38 may be discontinuous so as not to completely divide the passageway 22. Periodic gaps in the internal rib 38 allow for all gases to flow to the other side of the discontinuous internal rib 38 when one side of the passageway 22 may become pinched or blocked. In tubing 14 that has a continuous internal wall 38, pinching or blocking of one side of the passageway 22a or 22b may lead to uneven pressure distribution of the gas causing improper administration.

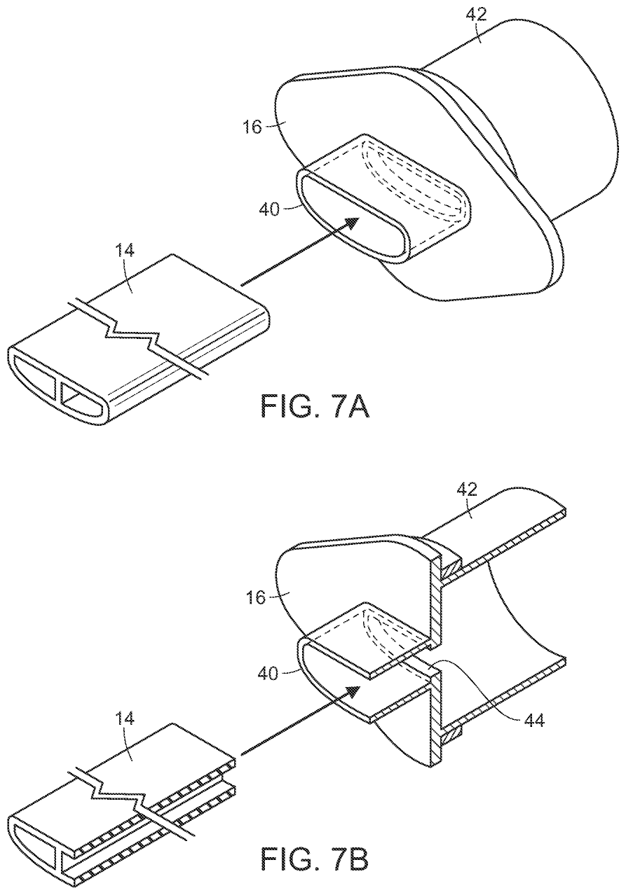

The supply adapter 16, various embodiments shown in detail in FIGS. 7A-7B, generally comprises a connector or coupling with a tube port 40 and a supply port 42 oppositely disposed. The tube port 40 preferably has a semi-circular cross-section that matches the semi-circular cross-section of the airway tubing 14. An opening 44 fluidly connects the tube port 40 to the supply port 42. Preferably, the opening 44 also has a semi-circular shape to match the shape of the tube port 40 and the airway tube 14. The supply port 42 should have a shape to match the connection available on gas supply tube 50 as may be found in a hospital or medical facility as from a ventilator or similar machine, to which the supply port 42 is configured to connect.

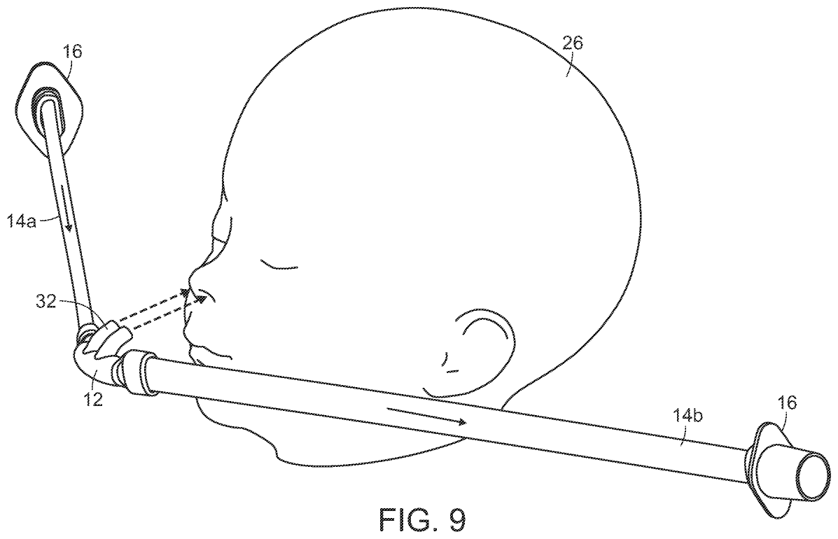

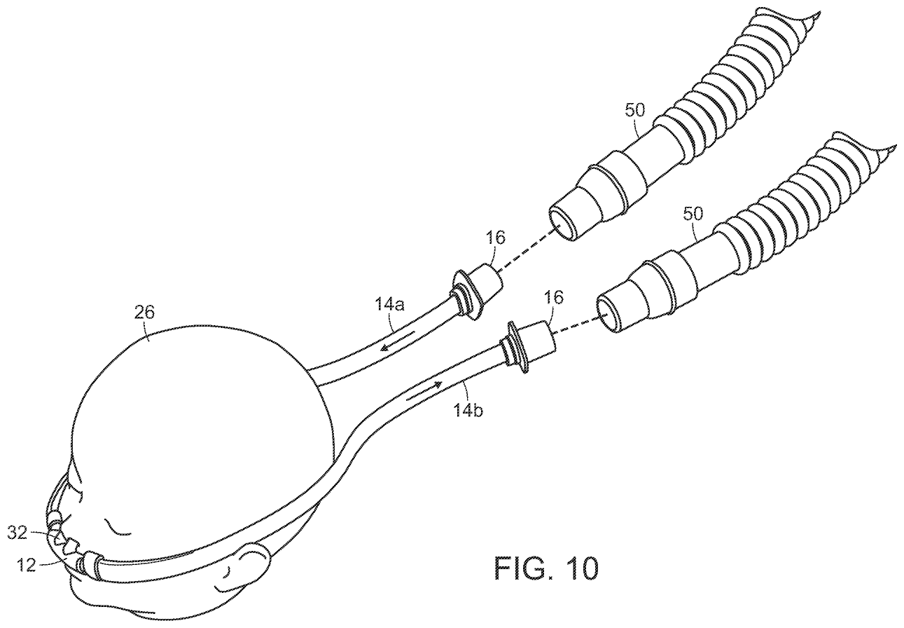

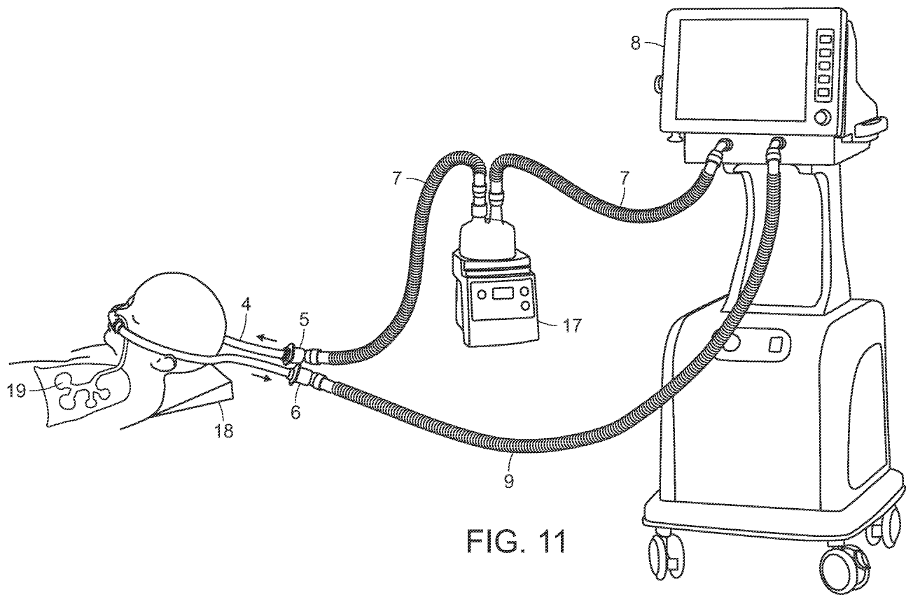

This embodiment of supply adapter 16 may be used in the system 10 in multiple configurations. A single supply adapter 16 can be connected to a single length of airway tubing 14 that is in turn connected to a nasal body that is closed at the opposite end. The single supply adapter 16 is then connected to a gas supply that provides gas for administration to a patient. Alternatively, two supply adapters 16 can be separately connected to different lengths of airway tubing 14, which separate lengths of airway tubing 14 are in turn connected to opposite ends of a nasal body 12. Each separate supply adapter 16 is connected to appropriately positioned gas supply connections to provide gas for administration to a patient. This configuration of two separate supply adapters 16, as shown in FIGS. 1, 8, 9 and 10, is referred to as an open-ended configuration 46.

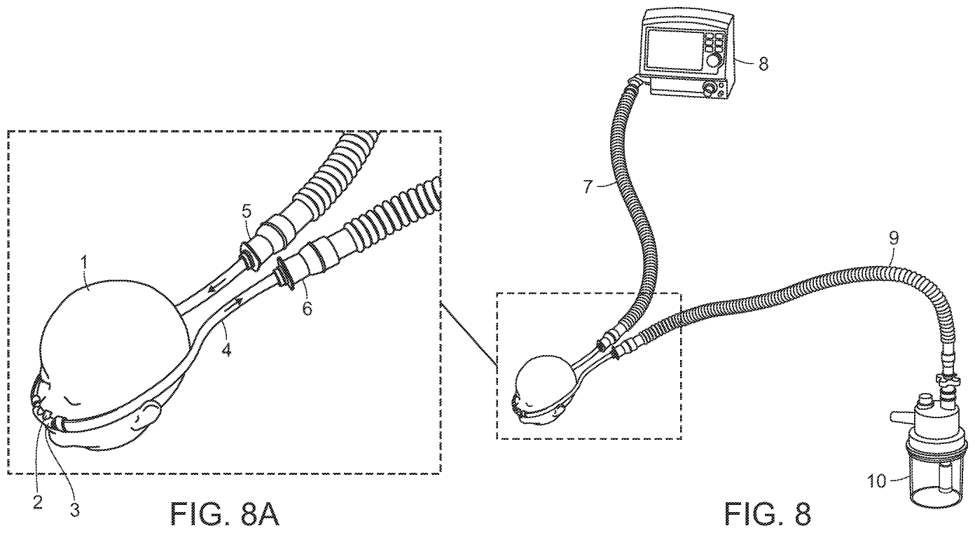

In a particular preferred embodiment, shown in FIGS. 8, 11, and 12, the nasal cannula system 10 is used in a ventilation system 70. Particularly, in FIGS. 8 and 8A, the ventilation system 70 includes a ventilator 72 connected by a gas supply tube 50a to an inhalation side airway tube 14a of the nasal cannula system 10. The exhalation side airway tube 14b on the nasal cannula system 10 is connected to a second gas supply tube 50b that is in turn connected to a bubble chamber 74. This ventilator system 70 incorporating the bubble chamber 74 achieves positive pressure in the circuit by effectively immersing the expiratory path in a water column to a desired depth to achieve the desired pressure resistance to flow.

FIG. 11 illustrates an alternate embodiment of the ventilation system 70 wherein the inhalation airway tube 14a and the exhalation tube 14b are both connected to a respective outlet 76a and inlet 76b of a large ventilator 76. A heater/humidifier 78 is preferably included in-line with the gas supply tube 50a on the inhalation side. This ventilator system 70 having the airway tubes 14a, 14b connected to the same ventilator 76 achieves positive pressure in the circuit by utilizing a valve adjustment in the outlet 76a or inlet 76b. As expected, the heater/humidifier 78 functions to condition the air in the inhalation airway tube 14a, i.e., add heat or humidity to the air, prior to introducing the air to the patient's nares.

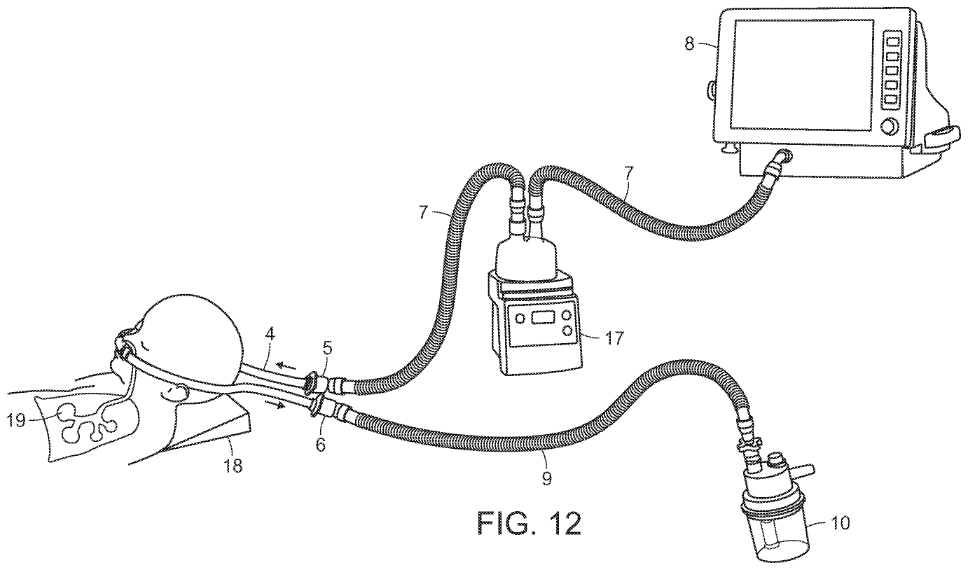

FIG. 12 illustrates yet another embodiment of the ventilation system 70 that is configured similarly to that shown in FIG. 11. Distinguished from the embodiment of FIG. 11, the system includes a ventilator 72 having a single outlet 76a. The gas supply tube 50b on the exhalation side is connected to a bubble chamber 74. This system 70 functions similarly to the system described in FIG. 8 above.

The nasal cannula system 10, gas airway tubing 14, and ventilator systems 70 described herein have a number of particular features that should preferably be employed in combination without departure from the scope and spirit of the invention.

Although preferred embodiments have been described in detail for purposes of illustration, various modifications may be made without departing from the scope and spirit of the invention. Accordingly, the invention is not to be limited, except as by the appended claims.

What is claimed is:

1. A nasal cannula system, comprising:
a nasal prong having nare tubes ending in nare ports configured for insertion into nostrils of a patient;
a first airway limb attached to a first connector on the nasal prong and in fluid communication with the nare tubes;
a second airway limb attached to a second connector on the nasal prong and in fluid communication with the nare tubes;
wherein the first airway limb comprises an inhalation tube on a ventilator system; and
wherein the second airway limb comprises an exhalation tube on the ventilator system.

2. The nasal cannula system of claim 1, wherein the inhalation tube is operationally connected to a positive pressure port on a ventilator.

3. The nasal cannula system of claim 2, wherein the exhalation tube is operationally connected to a back pressure device configured to maintain continuous positive pressure in the nasal cannula system.

4. The nasal cannula system of claim 3, wherein the back pressure device is a bubble chamber.

5. The nasal cannula system of claim 3, wherein the back pressure device is a variable control valve port on the ventilator.

6. The nasal cannula system of claim 2, further comprising a heater-humidifier fluidly disposed in the inhalation tube between the ventilator and the nasal prong.

7. The nasal cannula system of claim 1, further comprising a wedge elevating pad configured for disposal underneath a patient wearing the nasal prong.

8. The nasal cannula system of claim 1, wherein the first airway limb and the second airway limb both have a generally flat side joined to a generally curved side that together form a passageway therebetween having a semi-circular cross-section; and an internal rib extending along a length of the passageway, spanning from the generally flat side to the generally curved side.

9. The nasal cannula system of claim 8, wherein the internal rib is continuous along the length of the passageway so as to divide each of the first airway limb and the second airway limb into two separate passageways.

10. The nasal cannula system of claim 8, wherein the internal rib is discontinuous along the length of the passageway so as to allow fluid communication between regions of the passageway on opposite sides of the internal rib.

11. The nasal cannula system of claim 1, wherein the first airway limb and the second airway limb both have a generally flat exterior side configured to lay against a patient's skin when worn.
